(19) United States
(12) Patent Application Publication
(10) Pub. No.: US 2025/0352291 A1
(43) Pub. Date: Nov. 20, 2025

(54) ISOLATING APPARATUSES FOR MEDICAL PROCEDURES

(71) Applicant: Ultraview Medical, LLC, Carbondale, CO (US)

(72) Inventor: Victoria Powell, Basalt, CO (US)

(21) Appl. No.: 18/910,247

(22) Filed: Oct. 9, 2024

Related U.S. Application Data
(60) Provisional application No. 63/647,716, filed on May 15, 2024.

(51) Int. Cl. A61B 46/00; A41D 13/12; A61B 46/20; A61G 13/12

(52) U.S. Cl. CPC A61B 46/30; A41D 13/1254; A61B 46/20; A61G 13/123

(57) ABSTRACT

A body part isolating apparatus that includes a drape and an aperture in the drape for permitting a patient's body part to be extended therethrough to isolate the patient's body part for a medical procedure. The isolating apparatus can also include a support member positioned adjacent to the aperture for supporting the patient's body part extended through the aperture. A body part isolating apparatus that is a pair of shorts and includes an aperture in the shorts for permitting a medical professional access to a patient's body part for a medical procedure.

### STATEMENT REGARDING FEDERALLY SPONSORED RESEARCH OR DEVELOPMENT

Not applicable.

### CROSS-REFERENCE TO RELATED APPLICATIONS

The present application is a conversion of U.S. Provisional application having U.S. Ser. No. 63/647,716, filed May 15, 2024, which claims the benefit under 35 U.S.C. 119(e), the disclosures of which are hereby expressly incorporated herein by reference.

### BACKGROUND OF THE DISCLOSURE

### 1. Field of the Invention

The present disclosure relates to a body part isolating apparatus, such as drapes and garments, to allow for more efficient and sterile examination of a patient's scrotum, or other private body parts, while providing better privacy and comfort for the patient and physician during medical procedures and surgeries. The body part isolating apparatuses also yield better results for physicians due to improved screening conditions, which lead to better images.

### 2. Description of the Related Art

Medical drapes and garments are an important way for medical personnel to isolate a specific area of a patient's body. Common types of drapes and garments cover the patient's body, or a portion of the patient's body, during the procedure and maintain a sterile barrier around the isolated area. Such drapes and garments have been provided with a fenestration at a particular location and of a size particularly suited for access to the isolated area.

Known drapes and garments are not entirely satisfactory for the range of applications in which they are employed. For example, existing drapes and garments do not have fenestrations specific for the scrotal area of a patient. In addition, even conventional drapes and garments with a fenestration lack support for the patient's scrotal area making it difficult to access the scrotal area during a medical procedure, imaging, or surgery.

Thus, there exists a need for drapes and garments that improve upon and advance the design of known drapes and garments. Examples of new and useful drapes and garments relevant to the needs existing in the field are discussed below.

### SUMMARY OF THE INVENTION

The present disclosure is directed to a body part isolating apparatus that includes a drape and an aperture in the drape for permitting a patient's body part to be extended therethrough to isolate the patient's body part for a medical procedure. The isolating apparatus can also include a support member positioned adjacent to the aperture for supporting the patient's body part extended through the aperture.

The present disclosure is also directed to a body part isolating apparatus that is a pair of shorts and includes an aperture in the shorts for permitting a medical professional access to a patient's body part for a medical procedure.

### BRIEF DESCRIPTION OF THE DRAWINGS

FIG. 1A is a front elevation view of a body part isolating apparatus constructed in accordance with the present disclosure.

FIG. 1B is a front elevation view of the body part isolating apparatus laid over a patient.

FIG. 2 is a close-up view of the body part isolating apparatus shown in FIG. 1A constructed in accordance with the present disclosure.

FIGS. 3-5 are various views of tray portion of the body part isolating apparatus constructed in accordance with the present disclosure.

FIGS. 6A and 6B are front elevation views of another embodiment of the body part isolating apparatus constructed in accordance with the present disclosure.

FIG. 7 is a close-up view of the body part isolating apparatus shown in FIGS. 6A and 6B constructed in accordance with the present disclosure.

FIG. 8A is a front elevation view of a back side of a medical garment constructed in accordance with the present disclosure.

FIG. 8B is a side elevation view of the medical garment shown in FIG. 8A.

FIGS. 9A and 9B are front elevation views of another embodiment of the isolating apparatus constructed in accordance with the present disclosure.

FIGS. 10A and 10B are front elevation views of another embodiment of the isolating apparatus constructed in accordance with the present disclosure.

### DETAILED DESCRIPTION OF THE DISCLOSURE

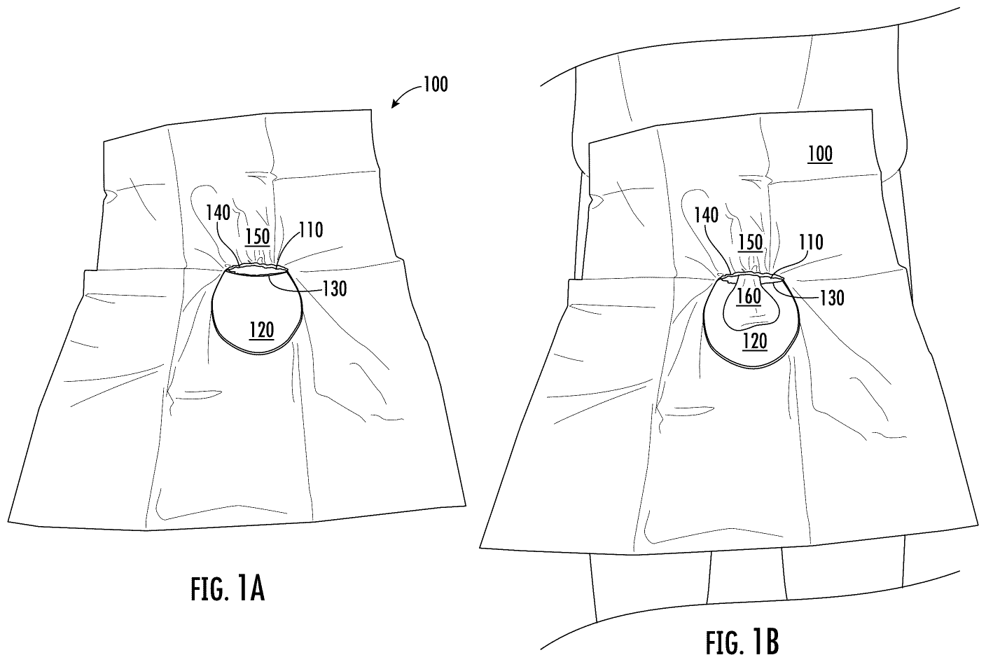

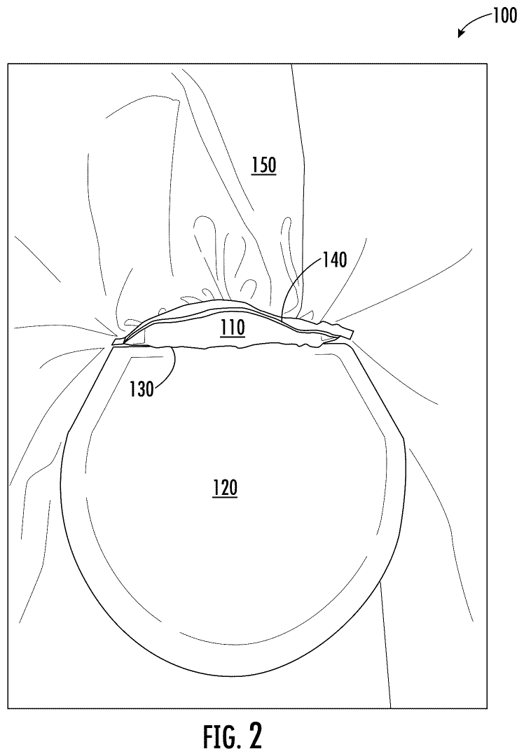

The present disclosure relates to a body part isolating apparatus 100, such as drapes and garments, to allow for more efficient and sterile examination of a patient's body parts, such as the scrotum, penis, vagina and anus, while providing a bit more privacy for the patient during medical procedures and surgeries. Referring now to FIGS. 1A-2, the apparatus 100 can be a drape and includes an aperture 110, a support member 120, an aperture bottom 130, and an aperture top 140. In other examples, the isolating apparatus 100 includes additional or alternative features, such as a second aperture located at secondary area 150. In some embodiments, the dimensions of the isolating apparatus 100 may be similar to a standard medical drape used in medical offices which would cover the patient's lap area. In other embodiments, the dimensions of the isolating apparatus 100 may be larger, such as covering most of the patient's body. The dimensions of the aperture can change to accommodate differing sizes of body parts and examination situations.

As can be seen in FIGS. 1A-2, aperture 110 functions as an opening in the isolating apparatus 100 for the patient's scrotum 160 to pass through the isolating apparatus 100. The aperture 110 serves to facilitate an efficient and modest isolation of the patient's scrotum 160. The aperture top 140 and the aperture bottom 130 define the aperture 110. The dimensions of the aperture 110 allow for the patient's scrotum 160 to pass through comfortably and efficiently.

The support member 120 functions to provide a platform to stabilize the patient's scrotum 160. Support member 120 can be coupled to the isolating apparatus 100 generally at the aperture bottom 130. The composition of the support member 120 may be of any number of rigid, semi-rigid, or flexible material or materials. In one embodiment, the composition of the support member 120 comprises a thick paper tray. In other embodiments, the support member 120 comprises a soft, yet rigid plastic material.

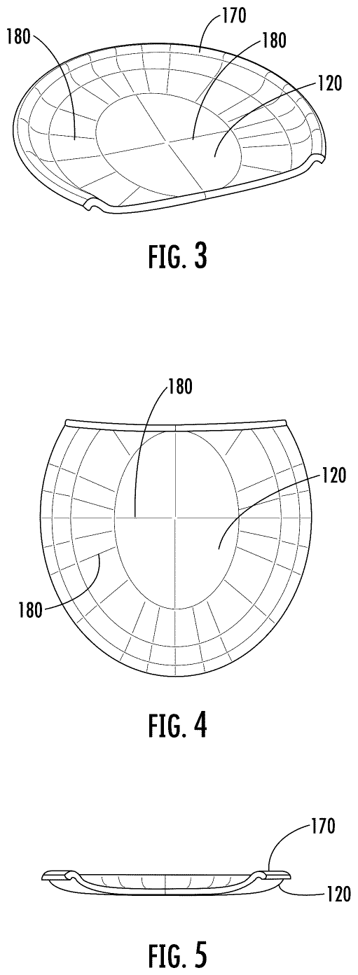

As shown in FIGS. 3-5, the shape of the support member 120 functions to stabilize the patient's scrotum 160 and can have a slight concave shape. The slight concave shape of the support member 120 prevents excessive shifting of the patient's scrotum 160 during the procedure. An edge 170 of the support member 120 also functions to prevent excessive shifting as well as capture any fluids used during the procedure or expelled from the patient. Line markings 180 can be disposed on the support member 120 to provide guidance for the medical professional in positioning the patient's scrotum 160 for the procedure.

In another embodiment, the support member 120 can be releasably attached to the isolating apparatus 100 such that various types and sizes of support members 120 could be used with the isolating apparatus 100. The support member 120 could be releasably secured to the isolating apparatus 100 in any manner known in the art. The support member 120 could be releasably secured to the isolating apparatus 100 with snaps, hook and loop strips, etc. Different size support members 120 accommodate different sizes of scrotums and different shapes and configurations accommodate different medical procedures and uses of the isolating apparatus 100. The dimensions of the aperture 110 are designed to accommodate the largest removeable support members 120 as well as the smallest removeable support members 120 without compromising the scrotal stabilizing function of the removable support member 120.

The isolating apparatus 100 can also be designed such that the support member 120 could also be positioned on any side of the aperture 110 to permit a medical professional access to whatever side of the scrotum 160 is desirable for a particular medical procedure. In one embodiment, the support member 120 could extend all the way around the aperture 110 to allow the medical professional to position the scrotum 160 at any desired position.

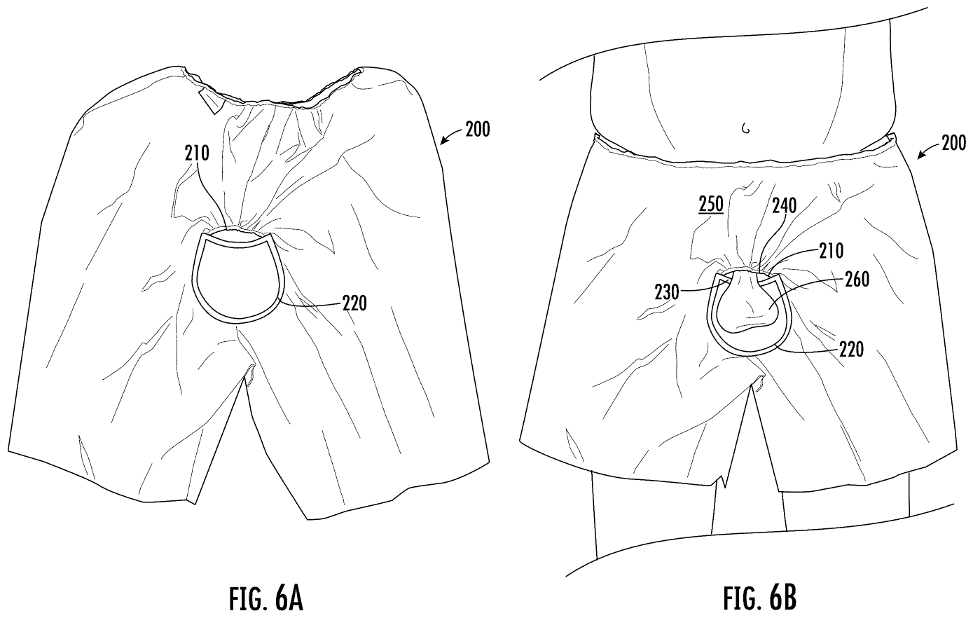

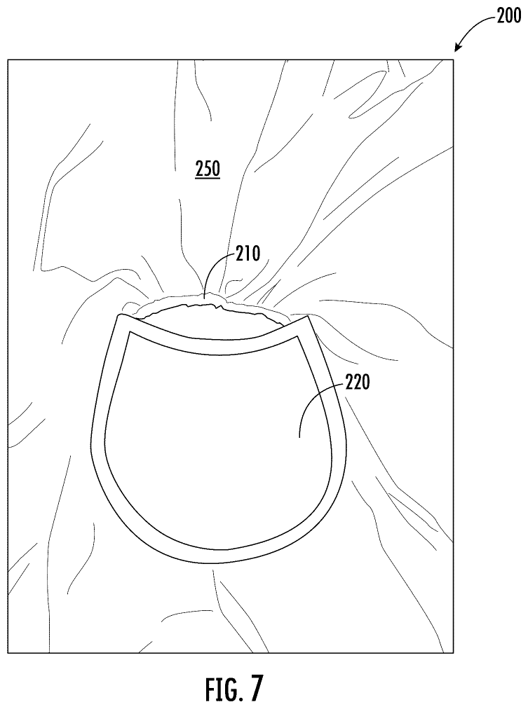

Referring now to FIGS. 6A-7, shown therein is another embodiment of a body part isolating apparatus 200. The isolating apparatus 200 in this embodiment is depicted as a garment that can be worn by a patient. More specifically, the isolating apparatus 200 is a pair of shorts that can provide a patient with even more coverage and provide more privacy. Similar to the drape version of the isolating apparatus 100, the isolating apparatus 200 includes an aperture 210, a support member 220, an aperture bottom 230, and an aperture top 240. In other examples, the isolating apparatus 200 includes additional or alternative features, such as a second aperture located at secondary area 250.

As can be seen in FIGS. 6A-7, the aperture 210 functions as an opening in the isolating apparatus 200 for the patient's scrotum 260 to pass through the isolating apparatus 200. The aperture 210 serves to facilitate an efficient and modest isolation of the patient's scrotum 260. The aperture top 240 and the aperture bottom 230 define the aperture 210. The dimensions of the aperture 210 allow for the patient's scrotum 260 to pass through comfortably and efficiently.

The support member 220 functions to provide a platform to stabilize the patient's scrotum 260. Support member 220 can be coupled to the isolating apparatus 200 generally at the aperture bottom 230. The composition of the support member 220 may be of any number of rigid, semi-rigid, or flexible material or materials. In one embodiment, the composition of the support member 220 comprises a thick paper tray. In other embodiments, the support member 220 comprises a soft, yet rigid plastic material. The support member 220 can be releasably attached to the isolating apparatus 200 and designed similar to the support member 120 shown in FIGS. 3-5 and described above.

The isolating apparatus 200 can also be designed such that the support member 220 could also be positioned on any side of the aperture 210 to permit a medical professional access to whatever side of the scrotum 260 is desirable for a particular medical procedure. In one embodiment, the support member 220 could extend all the way around the aperture 210 to allow the medical professional to position the scrotum 260 at any desired position.

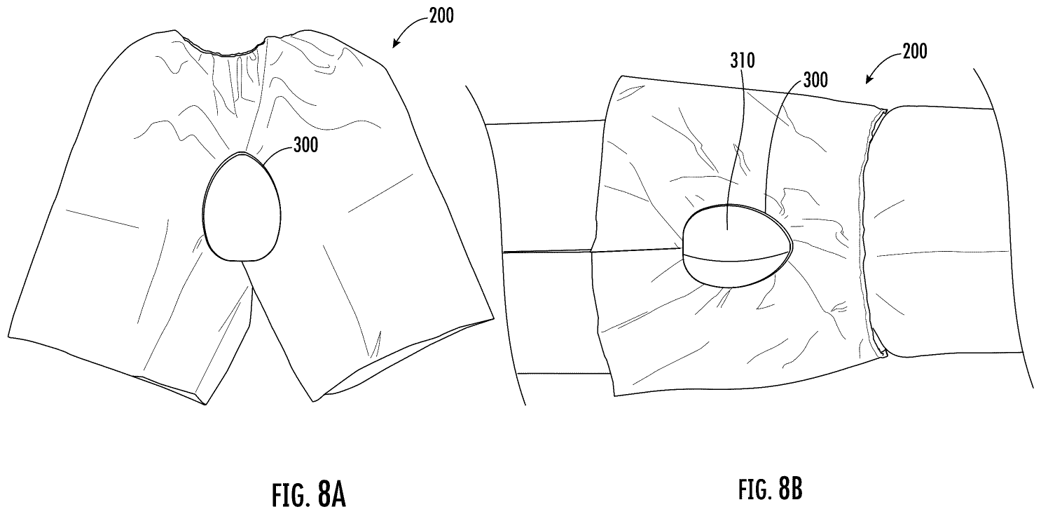

Referring now to FIGS. 8A and 8B, the isolating apparatus 200 can also include a rear opening 300 for permitting access to a patient's anus (not shown) or buttocks 310 during certain procedures, such as a colonoscopy or prostate exam. The rear opening 300 can be sized such that it is big enough to accommodate the necessary equipment and hands of the surgical team. The rear opening 300 allows access to a patient's anus or buttocks 310 while the remaining part of the isolating apparatus 200 covers the other private parts of the patient. It should be understood and appreciated that the isolating apparatus 200 can only include the rear opening 300 in certain embodiments and does not include the aperture 220 or support member 220 in the front of the isolating apparatus.

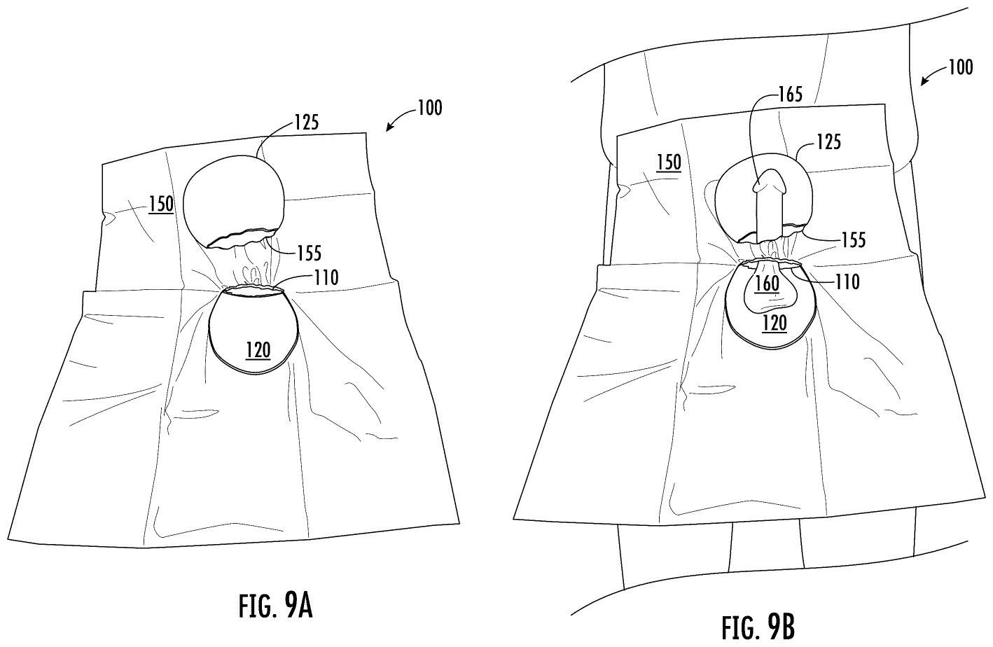

Referring now to FIGS. 9A and 9B, the secondary area 150 of the isolating apparatus 100 can also include a second aperture 155 for allowing access and isolating the penis 165 of a patient for a medical procedure. The isolating apparatus 100 can also include a second support member 125 for providing a stable surface for the penis 165 to be placed on during a medical procedure for dealing with the penis 165. The second support member 125 can be placed on any side of the second aperture 155 such that the penis 165 can be positioned in whatever orientation is necessary to accomplish the medical procedure. In one embodiment, the second support member 125 could extend all the way around the second aperture 155 so that the medical professional can have a stable surface for the penis 165 regardless of what side of the penis 165 the medical professional needs access to.

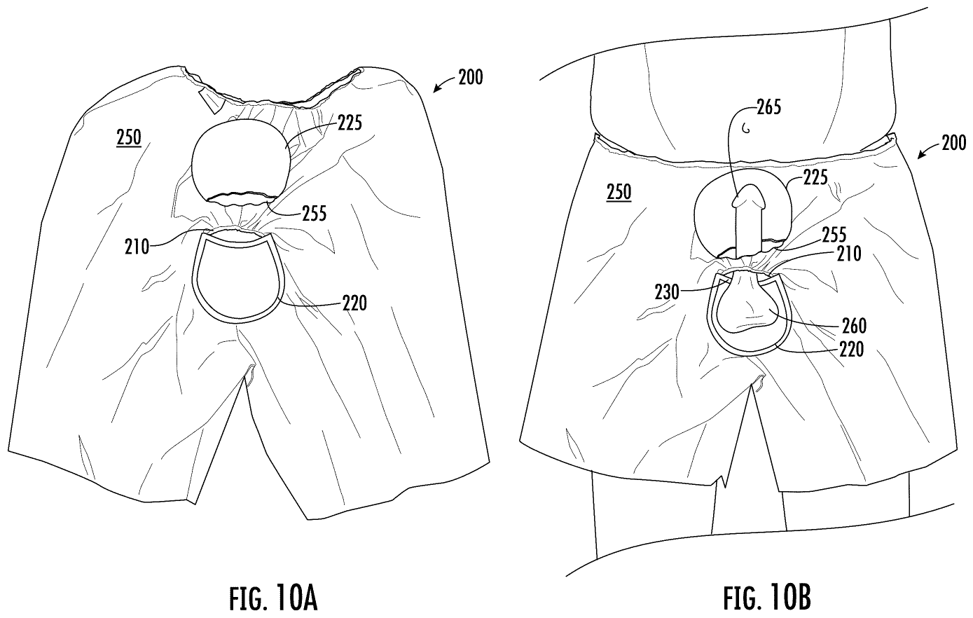

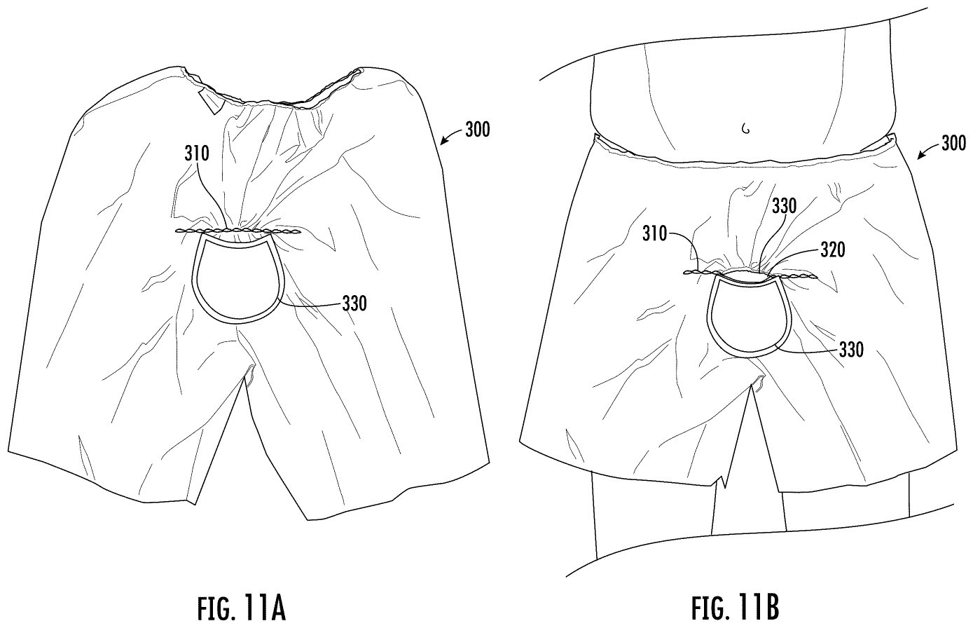

Referring now to FIGS. 10A and 10B, the secondary area 250 of the isolating apparatus 200 can also include a second aperture 255 for allowing access and isolating the penis 265 of a patient for a medical procedure. The isolating apparatus 200 can also include a second support member 225 for providing a stable surface for the penis 265 to be placed on during a medical procedure for dealing with the penis 265. The second support member 225 can be placed on any side of the second aperture 255 such that the penis 265 can be positioned in whatever orientation is necessary to accomplish the medical procedure. In one embodiment, the second support member 225 could extend all the way around the second aperture 255 so that the medical professional can have a stable surface for the penis 265 regardless of what side of the penis 265 the medical professional needs access to.

Referring now to FIGS. 11A and 11B, any of the apertures disclosed herein could created by the isolating apparatus 300 having a weakened portion 310 that is easily manipulated to cause the aperture 320 to appear. The embodiment shown in FIG. 6A is used as an illustration. For example, the weakened area 310 of the isolating apparatus 300 could be perforated so that the garment or drape is without an opening until desired by the patient or the medical professional. The aperture 320 could also come with a drawstring to keep the aperture 320 closed until the patient or medical professional desires. This embodiment could also include a support member 330 as described herein.

From the above description, it is clear that the present disclosure is well-adapted to carry out the objectives and to attain the advantages mentioned herein as well as those inherent in the disclosure. While presently preferred embodiments have been described herein, it will be understood that numerous changes may be made which will readily suggest themselves to those skilled in the art and which are accomplished within the spirit of the disclosure and claims.

1. A body part isolating apparatus, the isolating apparatus comprising:
a drape;
an aperture in the drape for permitting a patient's body part to be extended therethrough to isolate the patient's body part for a medical procedure; and
a support member positioned adjacent to the aperture for supporting the patient's body part extended through the aperture.

2. The isolating apparatus of claim 1 wherein the support member is releasably attached to the drape.

3. The isolating apparatus of claim 2 wherein the support member is a tray with an upturned edge around at least a portion of the tray.

4. The isolating apparatus of claim 1 wherein the patient's body part is a scrotum, penis or vagina.

5. The isolating apparatus of claim 1 further comprising a second aperture disposed therein to permit a second body part to be extended therethrough to be isolated for a medical procedure.

6. The isolating apparatus of claim 5 further comprising a second support member disposed adjacent to the second aperture for supporting the second body part for the medical procedure.

7. The isolating apparatus of claim 3 wherein the tray includes line markings thereon for use or guidance during the medical procedure.

8. The isolating apparatus of claim 1 wherein the aperture includes elastic to keep the aperture at least partially closed when a body part is not extended therethrough.

9. A body part isolating apparatus, the body part isolating apparatus comprising:
a pair of shorts;
an aperture in the pair of shorts for permitting a medical professional access to a patient's body part for a medical procedure; and
a rigid support member positioned adjacent to the aperture without covering the aperture, the rigid support member supports the patient's body part extended through the aperture.

10. The isolating apparatus of claim 9 wherein the aperture is a rear aperture in a backside of the short to permit the medical professional access to a patient's anus.

11. The body part isolating apparatus of claim 9 wherein the aperture in the pair of shorts is in a front side for permitting a patient's body part to be extended therethrough to isolate the patient's body part for a medical procedure.

12. (canceled)

13. The body part isolating apparatus of claim 1 wherein the rigid support member is releasably attached to the pair of shorts.

14. The body part isolating apparatus of claim 13 wherein the rigid support member is a tray with an upturned edge around at least a portion of the tray.

15. The body part isolating apparatus of claim 11 wherein the patient's body part is a scrotum or penis.

16. The isolating apparatus of claim 12 further comprising a second aperture disposed therein to permit a second body part to be extended therethrough to be isolated for a medical procedure.

17. The isolating apparatus of claim 16 further comprising a second support member disposed adjacent to the second aperture for supporting the second body part for the medical procedure.

18. The body part isolating apparatus of claim 14 wherein the tray includes line markings thereon for use or guidance during the medical procedure.

19. The isolating apparatus of claim 11 wherein the aperture includes elastic to keep the aperture at least partially closed when a body part is not extended therethrough.

20. The isolating apparatus of claim 9 wherein the aperture is created by manipulating a weakened area in the isolating apparatus.
